(19) United States
(12) Patent Application Publication
(10) Pub. No.: US 2025/0037798 A1
(43) Pub. Date: Jan. 30, 2025

(54) GENERATIVE LANGUAGE MODELS AND RELATED ASPECTS FOR PEPTIDE AND PROTEIN SEQUENCE DESIGN

(71) Applicant: THE JOHNS HOPKINS UNIVERSITY, Baltimore, MD (US)

(72) Inventors: Jeffrey RUFFOLO, Baltimore, MD (US); Richard SHUAI, Baltimore, MD (US); Jeffrey J. GRAY, Baltimore, MD (US)

(21) Appl. No.: 18/715,967

(22) PCT Filed: Dec. 7, 2022
(86) PCT No.: PCT/US2022/052178

§ 371 Date: Jun. 3, 2024

Related U.S. Application Data
(60) Provisional application No. 63/386,274, filed on Dec. 6, 2022.
(60) Provisional application No. 63/287,204, filed on Dec. 8, 2021.

(51) Int. Cl. G16B 35/10; G16B 40/20

(52) U.S. Cl. CPC G16B 35/10; G16B 40/20

(57) ABSTRACT

Provided herein are methods of producing a trained model for generating peptide or protein sequence information and infilling of targeted residue spans. In some embodiments, the methods include training a model using a training dataset comprising a population of reference amino acid sequence representations in which a given amino acid sequence representation in the population is conditioned on one or more conditioning tags that provide a controllable generation of selected amino acid sequence representation types. Related methods, systems, and computer program products are also provided.

### CROSS-REFERENCE TO RELATED APPLICATIONS

This application is the national stage entry of International Patent Application No. PCT/US2022/052178, filed on Dec. 7, 2022, and published as WO 2023/107580 A1 on Jun. 15, 2023, which claims the benefit of U.S. Provisional Patent Application Ser. Nos. 63/287,204, filed on Dec. 8, 2021 and 63/386,274, filed on Dec. 6, 2022, which are hereby incorporated by reference in their entireties.

### STATEMENT REGARDING FEDERALLY SPONSORED RESEARCH OR DEVELOPMENT

This invention was made with government support under grant numbers GM078221 and GM141881, awarded by the National Institutes of Health, and under grant numbers 1659649 and 1950697, awarded by the National Science Foundation. The government has certain rights in the invention.

### BACKGROUND

Antibodies have become popular for therapeutics because of their diversity and ability to bind antigens with high specificity. Traditionally, monoclonal antibodies (mAbs) have been obtained using hybridoma technology, which involves the immunization of animals. In 1985, the development of phage display technology allowed for in vitro selection of specific, high-affinity mAbs from large antibody libraries. Despite such advances, therapeutic mAbs derived from display technologies face issues with developability, such as poor expression, low solubility, low thermal stability, and high aggregation. Display technologies rely on a high-quality and diverse antibody library as a starting point to isolate high-affinity antibodies that are more developable. Synthetic antibody libraries are prepared by introducing synthetic DNA into regions of the antibody sequences that define the complementarity determining regions (CDRs), allowing for man-made antigen-binding sites. However, the space of possible synthetic antibody sequences is very large (diversifying 10 positions of a CDR yields 20¹⁰-10¹³ possible variants). To discover antibodies with high affinity, massive synthetic libraries on the order of 10¹⁰-10¹¹ variants must be constructed, often containing substantial fractions of non-functional antibodies.

Recent work has leveraged natural language processing methods for unsupervised pre-training on massive databases of raw protein sequences for which structural data is unavailable. These works have explored explored a variety of pre-training tasks and downstream model applications. For example, the ESM family of models (trained for masked language modeling) have been applied to representation learning, variant effect prediction, and protein structure prediction. Autoregressive language modeling, an alternative paradigm for pre-training, has also been applied to protein sequence modeling. Such models have been shown to generate diverse protein sequences, which often adopt natural folds despite significantly divergent residue makeup. In some cases, these generated sequences even retain enzymatic activity comparable to natural proteins. Autoregressive language models have also been shown to be powerful zero-shot predictors of protein fitness, with performance in some cases continuing to improve with model scale.

Another set of language models have been developed specifically for antibody-related tasks. The majority of prior work in this area has focused on masked language modeling of sequences in the Observed Antibody Space (OAS) database. Prihoda et al. (A platform for antibody design, humanization and humanness evaluation based on natural antibody repertoires and deep learning. bioRxiv, 2021) developed Sapiens, a pair of distinct models (each with 569K parameters) for heavy and light chain masked language modeling. The Sapiens models were trained on 20M and 19M heavy and light chains respectively, and shown to be effective tools for antibody humanization. Ruffolo et al. (Deciphering antibody affinity maturation with language models and weakly supervised learning. arXiv preprint arXiv:2112.07782, 2021) developed AntiBERTy, a single masked language model (26M parameters) trained on a corpous of 558M sequences, including both heavy and light chains. AntiBERTy has been applied to representation learning for protein structure prediction. Leem et al. (Deciphering the language of antibodies using self-supervised learning, Patterns, Volume 3, Issue 7, 2022) developed AntiBERTa, a single masked language model (86M parameters) trained on a corpus of 67M antibody sequences (both heavy and light). Representations for AntiBERTa were used for paratope prediction. Olsen et al. (Ablang: An antibody language model for completing antibody sequences. bioRxiv, 2022) developed AbLang, a pair of masked language models trained on 14M heavy chains and 187K light chains, for sequence restoration. For sequence generation, autoregressive generative models have been trained on nanobody sequences and used for library design. Shin et al. (Protein design and variant prediction using autoregressive generative models. Nature communications, 12(1):1-11, 2021) experimentally validated a set of generated nanobody sequences with generated CDR3 loops and showed promising improvements to viability and binding discovery when compared to traditional approaches, despite being over 1000-fold smaller. However, because this generative model was unidirectional, it could not be used to directly re-design the CDR3 loop within, and instead had to be oversampled to produce sequences matching the residues following the loop.

Accordingly, there is a need for additional techniques for generating libraries of synthetic antibody sequences and related models for antibody discovery and optimization.

### SUMMARY

The present disclosure relates, in certain aspects, to methods of producing a trained bidirectional generative immunoglobulin language model (IgLM). In some aspects, the present disclosure also provides methods of generating a library of synthetic antibody sequences using the IgLM. Related systems and computer readable media are also provided. In some embodiments, for example, the present disclosure provides what is sometimes referred to herein as an Immunoglobulin Language Model (IgLM), which leverages bidirectional context for designing antibody sequence spans of varying lengths while training on a large-scale natural antibody dataset. We show that IgLM can generate full-length antibody sequences conditioned on chain type and species-of-origin. Furthermore, IgLM can diversify loops on an antibody to generate high-quality libraries that display favorable biophysical properties while resembling human antibodies. IgLM is a powerful tool for antibody discovery and optimization. These and other attributes will be apparent upon complete review of the present disclosure, including the accompanying figures.

In one aspect, for example, the present disclosure provides a method of producing a trained bidirectional generative immunoglobulin language model (IgLM) using a computer. The method includes training, by the computer, the generative IgLM using a training dataset comprising a population of reference immunoglobulin (Ig) amino acid sequence representations, wherein a given reference Ig amino acid sequence representation in the population is represented as A=(a₁, . . . , aₙ), where at is an amino acid residue at position i of the given reference immunoglobulin amino acid sequence representation, wherein a [MASK] token of length m is applied to the given reference Ig amino acid sequence representation, wherein the [MASK] token comprises a starting amino acid residue position j from [1, n−m+1] in the given reference Ig amino acid sequence representation, and wherein at least one conditioning tag is applied to the given reference Ig amino acid sequence representation, thereby producing the trained bidirectional generative IgLM. In some embodiments, the [MASK] token replaces a CDR loop span in the given reference Ig amino acid sequence representation.

In another aspect, the present disclosure provides a method of generating a library of synthetic antibody sequences using a computer. The method includes receiving, by the computer, at least one test immunoglobulin (Ig) amino acid sequence representation into a trained bidirectional generative immunoglobulin language model (IgLM), wherein the test Ig amino acid sequence representation is represented as A=(a₁, . . . , aₙ), where at is an amino acid residue at position i of the test Ig amino acid sequence representation, wherein a [MASK] token of length m is applied to the given test Ig amino acid sequence representation, wherein the [MASK]token comprises a starting amino acid residue position j from [1, n−m+1] in the test Ig amino acid sequence representation, and wherein at least one conditioning tag is applied to the test Ig amino acid sequence representation, wherein the bidirectional generative IgLM is trained using a training dataset comprising a population of reference immunoglobulin (Ig) amino acid sequence representations, wherein a given reference Ig amino acid sequence representation in the population is represented as A=(a₁, . . . , aₙ), where at is an amino acid residue at position i of the given reference immunoglobulin amino acid sequence representation, wherein the [MASK] token of length m is applied to the given reference Ig amino acid sequence representation, wherein the [MASK] token comprises a starting amino acid residue position j from [1, n−m+1] in the given reference Ig amino acid sequence representation, and wherein the conditioning tag is applied to the given reference Ig amino acid sequence representation, and wherein the bidirectional generative IgLM infills amino acid residues replaced by the [MASK] token applied to the given test Ig amino acid sequence representation to generate the library of synthetic antibody sequences. In some embodiments, the [MASK] token replaces a CDR loop span in the test and reference Ig amino acid sequence representations. In some embodiments, the library of synthetic antibody sequences produced by the methods disclosed herein includes variable length sequences, whereas in other embodiments, the library of synthetic antibody sequences produced by the methods disclosed herein includes sequences of uniform length. The library of synthetic antibody sequences produced by the methods disclosed herein can be of essentially any size, such as from one sequence to millions of sequences or more (e.g., about 50 sequences, about 100 sequences, about 500 sequences, about 1000 sequences, about 5000 sequences, about 10000 sequences, about 50000 sequences, about 100000 sequences, about 500000 sequences, about 1000000 sequences, about 5000000 sequences, etc.).

In another aspect, the present disclosure provides a method of producing a trained model using a computer, the method comprising training, by the computer, a model using a training dataset comprising a population of reference amino acid sequence representations, wherein a given reference amino acid sequence representation in the population is represented as A=(a₁, . . . , aₙ), where at is an amino acid residue at position i of the given reference amino acid sequence representation, wherein a [MASK] token of length m is applied to the given reference amino acid sequence representation, wherein the [MASK] token comprises a starting amino acid residue position j from [1, n−m+1] in the given reference amino acid sequence representation, and wherein at least one conditioning tag is applied to the given reference amino acid sequence representation, thereby producing the trained model. In some embodiments, the population of reference amino acid sequence representations comprises a population comprises a population of reference immunoglobulin (Ig) amino acid sequence representations and wherein the trained model comprises a trained bidirectional generative immunoglobulin language model (IgLM).

In another aspect, the present disclosure provides a method of producing a trained bidirectional generative immunoglobulin language model (IgLM) using a computer, the method comprising training, by the computer, the generative IgLM using a training dataset comprising a population of reference immunoglobulin (Ig) amino acid sequence representations, wherein a given reference Ig amino acid sequence representation in the population is represented as A=(a₁, . . . , aₙ), where at is an amino acid residue at position i of the given reference immunoglobulin amino acid sequence representation, wherein a [MASK] token of length m is applied to the given reference Ig amino acid sequence representation, wherein the [MASK] token comprises a starting amino acid residue position j from [1, n−m+1] in the given reference Ig amino acid sequence representation, and wherein at least one conditioning tag is applied to the given reference Ig amino acid sequence representation, thereby producing the trained bidirectional generative IgLM.

In another aspect, the present disclosure provides a method of producing a trained model for generating peptide or protein sequence information and infilling of targeted residue spans using a computer, the method comprising training, by the computer, a model using a training dataset comprising a population of reference amino acid sequence representations, wherein a given amino acid sequence representation in the population is conditioned on one or more conditioning tags that provide a controllable generation of selected amino acid sequence representation types, thereby producing the trained model. In some embodiments, the method includes using the trained model to generate at least one infilled residue span at a selected position of a given amino acid sequence representation. In some embodiments, the method includes determining an infilling perplexity value of the infilled residue span to produce a determined infilling perplexity value. In some embodiments, the method includes comprising using the trained model to generate a library of infilled amino acid sequence representations that comprises one or more selected traits. In some embodiments, the selected traits comprise one or more developability traits. In some embodiments, the method includes synthesizing a peptide or a protein that corresponds to the given amino acid sequence representation. In some embodiments, the protein comprises a substantially full-length protein. In some embodiments, the peptide or the protein comprises a therapeutic peptide or a therapeutic protein. In some embodiments, the given amino acid sequence representation comprises an immunoglobulin (Ig) amino acid sequence representation and wherein the selected position comprises a complementary-determining region (CDR). In some embodiments, the population of reference amino acid sequence representations comprises a population comprises a population of reference immunoglobulin (Ig) amino acid sequence representations and wherein the conditioning tags comprise at least a chain-type tag (c_{c}) and at least a species-of-origin identifier tag (cₛ). In some embodiments, the selected amino acid sequence representation types comprise a chain-type and/or a species-of-origin type. In some embodiments, the method includes comprising using an autoregressive language modeling technique to train the model.

In another aspect, the present disclosure provides a system comprising at least one controller that comprises, or is capable of accessing, computer readable media comprising non-transitory computer executable instructions which, when executed by at least one electronic processor, perform at least: training a bidirectional generative immunoglobulin language model (IgLM) using a training dataset comprising a population of reference immunoglobulin (Ig) amino acid sequence representations, wherein a given reference Ig amino acid sequence representation in the population is represented as A=(a₁, . . . , aₙ), where at is an amino acid residue at position i of the given reference immunoglobulin amino acid sequence representation, wherein a [MASK] token of length m is applied to the given reference Ig amino acid sequence representation, wherein the [MASK] token comprises a starting amino acid residue position j from [1, n−m+1] in the given reference Ig amino acid sequence representation, and wherein at least one conditioning tag is applied to the given reference Ig amino acid sequence representation. In some embodiments, the [MASK]token replaces a CDR loop span in the reference Ig amino acid sequence representation.

In another aspect, the present disclosure provides a system comprising at least one controller that comprises, or is capable of accessing, computer readable media comprising non-transitory computer executable instructions which, when executed by at least one electronic processor, perform at least: receiving at least one test immunoglobulin (Ig) amino acid sequence representation into a trained bidirectional generative immunoglobulin language model (IgLM), wherein the test Ig amino acid sequence representation is represented as A=(a₁, . . . , aₙ), where at is an amino acid residue at position i of the test Ig amino acid sequence representation, wherein a [MASK] token of length m is applied to the given test Ig amino acid sequence representation, wherein the [MASK] token comprises a starting amino acid residue position j from [1, n−m+1] in the test Ig amino acid sequence representation, and wherein at least one conditioning tag is applied to the test Ig amino acid sequence representation, wherein the bidirectional generative IgLM is trained using a training dataset comprising a population of reference immunoglobulin (Ig) amino acid sequence representations, wherein a given reference Ig amino acid sequence representation in the population is represented as A=(a₁, . . . , aₙ), where at is an amino acid residue at position i of the given reference immunoglobulin amino acid sequence representation, wherein the [MASK] token of length m is applied to the given reference Ig amino acid sequence representation, wherein the [MASK] token comprises a starting amino acid residue position j from [1, n−m+1] in the given reference Ig amino acid sequence representation, and wherein the conditioning tag is applied to the given reference Ig amino acid sequence representation, and wherein the bidirectional generative IgLM infills amino acid residues replaced by the [MASK] token applied to the given test Ig amino acid sequence representation to generate a library of synthetic antibody sequences. In some embodiments, the [MASK] token replaces a CDR loop span in the test and reference Ig amino acid sequence representations.

In another aspect, the present disclosure provides a computer readable media comprising non-transitory computer executable instruction which, when executed by at least electronic processor, perform at least: training a bidirectional generative immunoglobulin language model (IgLM) using a training dataset comprising a population of reference immunoglobulin (Ig) amino acid sequence representations, wherein a given reference Ig amino acid sequence representation in the population is represented as A=(a₁, . . . , aₙ), where at is an amino acid residue at position i of the given reference immunoglobulin amino acid sequence representation, wherein a [MASK] token of length m is applied to the given reference Ig amino acid sequence representation, wherein the [MASK] token comprises a starting amino acid residue position j from [1, n−m+1] in the given reference Ig amino acid sequence representation, and wherein at least one conditioning tag is applied to the given reference Ig amino acid sequence representation. In some embodiments, the [MASK]token replaces a CDR loop span in the reference Ig amino acid sequence representation.

In another aspect, the present disclosure provides a computer readable media comprising non-transitory computer executable instruction which, when executed by at least electronic processor, perform at least: receiving at least one test immunoglobulin (Ig) amino acid sequence representation into a trained bidirectional generative immunoglobulin language model (IgLM), wherein the test Ig amino acid sequence representation is represented as A=(a₁, . . . , aₙ), where at is an amino acid residue at position i of the test Ig amino acid sequence representation, wherein a [MASK] token of length m is applied to the given test Ig amino acid sequence representation, wherein the [MASK] token comprises a starting amino acid residue position j from [1, n−m+1] in the test Ig amino acid sequence representation, and wherein at least one conditioning tag is applied to the test Ig amino acid sequence representation, wherein the bidirectional generative IgLM is trained using a training dataset comprising a population of reference immunoglobulin (Ig) amino acid sequence representations, wherein a given reference Ig amino acid sequence representation in the population is represented as A=(a₁, . . . , aₙ), where at is an amino acid residue at position i of the given reference immunoglobulin amino acid sequence representation, wherein the [MASK] token of length m is applied to the given reference Ig amino acid sequence representation, wherein the [MASK] token comprises a starting amino acid residue position j from [1, n−m+1] in the given reference Ig amino acid sequence representation, and wherein the conditioning tag is applied to the given reference Ig amino acid sequence representation, and wherein the bidirectional generative IgLM infills amino acid residues replaced by the [MASK] token applied to the given test Ig amino acid sequence representation to generate a library of synthetic antibody sequences. In some embodiments, the [MASK] token replaces a CDR loop span in the test and reference Ig amino acid sequence representations.

In some embodiments, the conditioning tag comprises a chain type, c_{c}, and/or a species-of-origin type, cₛ. In some embodiments, the chain type, c_{c}, comprises a heavy chain or a light chain. In some embodiments, the population of reference Ig amino acid sequence representations comprise monoclonal Ig amino acid sequence representations. In some embodiments, the methods disclosed herein include producing (e.g., synthesizing the amino acid sequences or nucleic acids encoding the same, expressing those nucleic acids in host organisms, or the like) a library of synthetic immunoglobulins that comprise one or more biochemical properties using the trained bidirectional generative IgLM. In some embodiments, the biochemical properties are selected from the group consisting of: a solubility level, a thermal stability level, an aggregation level, and an immunogenicity level.

In some embodiments, the [MASK] token replaces a span of amino acid residues represented as S=(aⱼ, . . . , aⱼ+m−1) in the given reference Ig amino acid sequence representation. In some embodiments, the bidirectional generative IgLM infills amino acid residues replaced by the [MASK] token. In some embodiments, the [MASK]token applied to the given reference Ig amino acid sequence representation forms a sequence representation A_{\s}=(a₁, . . . , aⱼ₋₁, [MASK], aⱼ₊ₘ, . . . , aₙ). In some embodiments, the given reference Ig amino acid sequence representation forms a sequence representation of (c_{c}, cₛ, a₁, . . . , aⱼ₋₁, [MASK] token; aⱼ₊ₘ, . . . , aₙ; [SEP] token, aⱼ, . . . , aⱼ₊ₘ₋₁, [ANS] token).

In another aspect, the present disclosure provides a system comprising at least one controller that comprises, or is capable of accessing, computer readable media comprising non-transitory computer executable instructions which, when executed by at least one electronic processor, perform at least: training a bidirectional generative immunoglobulin language model (IgLM) using a training dataset comprising a population of reference immunoglobulin (Ig) amino acid sequence representations, wherein a given reference Ig amino acid sequence representation in the population is represented as A=(a₁, . . . , aₙ), where at is an amino acid residue at position i of the given reference immunoglobulin amino acid sequence representation, wherein a [MASK] token of length m is applied to the given reference Ig amino acid sequence representation, wherein the [MASK] token comprises a starting amino acid residue position j from [1, n−m+1] in the given reference Ig amino acid sequence representation, and wherein at least one conditioning tag is applied to the given reference Ig amino acid sequence representation.

In another aspect, the present disclosure provides a computer readable media comprising non-transitory computer executable instruction which, when executed by at least electronic processor, perform at least: training a bidirectional generative immunoglobulin language model (IgLM) using a training dataset comprising a population of reference immunoglobulin (Ig) amino acid sequence representations, wherein a given reference Ig amino acid sequence representation in the population is represented as A=(a₁, . . . , aₙ), where at is an amino acid residue at position i of the given reference immunoglobulin amino acid sequence representation, wherein a [MASK] token of length m is applied to the given reference Ig amino acid sequence representation, wherein the [MASK] token comprises a starting amino acid residue position j from [1, n−m+1] in the given reference Ig amino acid sequence representation, and wherein at least one conditioning tag is applied to the given reference Ig amino acid sequence representation.

In another aspect, the present disclosure provides a computer readable media comprising non-transitory computer executable instruction which, when executed by at least electronic processor, perform at least: receiving at least one test immunoglobulin (Ig) amino acid sequence representation into a trained bidirectional generative immunoglobulin language model (IgLM), wherein the test Ig amino acid sequence representation is represented as A=(a₁, . . . , aₙ), where at is an amino acid residue at position i of the test Ig amino acid sequence representation, wherein a [MASK] token of length m is applied to the given test Ig amino acid sequence representation, wherein the [MASK] token comprises a starting amino acid residue position j from [1, n−m+1] in the test Ig amino acid sequence representation, and wherein at least one conditioning tag is applied to the test Ig amino acid sequence representation, wherein the bidirectional generative IgLM is trained using a training dataset comprising a population of reference immunoglobulin (Ig) amino acid sequence representations, wherein a given reference Ig amino acid sequence representation in the population is represented as A=(a₁, . . . , aₙ), where at is an amino acid residue at position i of the given reference immunoglobulin amino acid sequence representation, wherein the [MASK] token of length m is applied to the given reference Ig amino acid sequence representation, wherein the [MASK] token comprises a starting amino acid residue position j from [1, n−m+1] in the given reference Ig amino acid sequence representation, and wherein the conditioning tag is applied to the given reference Ig amino acid sequence representation, and wherein the bidirectional generative IgLM infills amino acid residues replaced by the [MASK] token applied to the given test Ig amino acid sequence representation to generate a library of synthetic antibody sequences.

In another aspect, the present disclosure provides a method of generating a library of synthetic peptide or protein sequences using a computer, the method comprising receiving, by the computer, at least one test amino acid sequence representation into a trained model, wherein the test amino acid sequence representation is represented as A=(a₁, . . . , aₙ), where at is an amino acid residue at position i of the test amino acid sequence representation, wherein a [MASK] token of length m is applied to the given test amino acid sequence representation, wherein the [MASK] token comprises a starting amino acid residue position j from [1, n−m+1] in the test amino acid sequence representation, and wherein at least one conditioning tag is applied to the test amino acid sequence representation, wherein the model is trained using a training dataset comprising a population of reference amino acid sequence representations, wherein a given reference amino acid sequence representation in the population is represented as A=(a₁, . . . , aₙ), where at is an amino acid residue at position i of the given reference amino acid sequence representation, wherein the [MASK] token of length m is applied to the given reference amino acid sequence representation, wherein the [MASK] token comprises a starting amino acid residue position j from [1, n−m+1] in the given reference amino acid sequence representation, and wherein the conditioning tag is applied to the given reference amino acid sequence representation, and wherein the model infills amino acid residues replaced by the [MASK] token applied to the given test amino acid sequence representation to generate the library of synthetic peptide or protein sequences.

In another aspect, the present disclosure provides a system comprising at least one controller that comprises, or is capable of accessing, computer readable media comprising non-transitory computer executable instructions which, when executed by at least one electronic processor, perform at least: training a model using a training dataset comprising a population of reference peptide or protein amino acid sequence representations, wherein a given reference amino acid sequence representation in the population is represented as A=(a₁, . . . , aₙ), where at is an amino acid residue at position i of the given reference amino acid sequence representation, wherein a [MASK] token of length m is applied to the given reference amino acid sequence representation, wherein the [MASK] token comprises a starting amino acid residue position j from [1, n−m+1] in the given reference Ig amino acid sequence representation, and wherein at least one conditioning tag is applied to the given reference amino acid sequence representation.

In another aspect, the present disclosure provides a system comprising at least one controller that comprises, or is capable of accessing, computer readable media comprising non-transitory computer executable instructions which, when executed by at least one electronic processor, perform at least: training a model using a training dataset comprising a population of reference amino acid sequence representations, wherein a given amino acid sequence representation in the population is conditioned on one or more conditioning tags that provide a controllable generation of selected amino acid sequence representation types.

In another aspect, the present disclosure provides a computer readable media comprising non-transitory computer executable instruction which, when executed by at least electronic processor, perform at least: training a model using a training dataset comprising a population of reference amino acid sequence representations, wherein a given reference amino acid sequence representation in the population is represented as A=(a₁, . . . , aₙ), where at is an amino acid residue at position i of the given reference amino acid sequence representation, wherein a [MASK] token of length m is applied to the given reference amino acid sequence representation, wherein the [MASK] token comprises a starting amino acid residue position j from [1, n−m+1] in the given reference amino acid sequence representation, and wherein at least one conditioning tag is applied to the given reference amino acid sequence representation.

In another aspect, the present disclosure provides a computer readable media comprising non-transitory computer executable instruction which, when executed by at least electronic processor, perform at least: receiving at least one test amino acid sequence representation into a trained model, wherein the test amino acid sequence representation is represented as A=(a₁, . . . , aₙ), where at is an amino acid residue at position i of the test amino acid sequence representation, wherein a [MASK] token of length m is applied to the given test amino acid sequence representation, wherein the [MASK] token comprises a starting amino acid residue position j from [1, n−m+1] in the test amino acid sequence representation, and wherein at least one conditioning tag is applied to the test amino acid sequence representation, wherein the model is trained using a training dataset comprising a population of reference amino acid sequence representations, wherein a given reference amino acid sequence representation in the population is represented as A=(a₁, . . . , aₙ), where at is an amino acid residue at position i of the given reference amino acid sequence representation, wherein the [MASK] token of length m is applied to the given reference amino acid sequence representation, wherein the [MASK] token comprises a starting amino acid residue position j from [1, n−m+1] in the given reference amino acid sequence representation, and wherein the conditioning tag is applied to the given reference amino acid sequence representation, and wherein the model infills amino acid residues replaced by the [MASK] token applied to the given test amino acid sequence representation to generate a library of synthetic amino acid sequences.

In another aspect, the present disclosure provides a computer readable media comprising non-transitory computer executable instruction which, when executed by at least electronic processor, perform at least: training a model using a training dataset comprising a population of reference amino acid sequence representations, wherein a given amino acid sequence representation in the population is conditioned on one or more conditioning tags that provide a controllable generation of selected amino acid sequence representation types.

### BRIEF DESCRIPTION OF THE DRAWINGS

The accompanying drawings, which are incorporated in and constitute a part of this specification, illustrate certain embodiments, and together with the written description, serve to explain certain principles of the methods, systems, and related computer readable media disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a schematic diagram of an exemplary system suitable for use with certain aspects disclosed herein.

FIGS. 2A-2D. Overview of IgLM model for antibody sequence generation. (A) IgLM is trained by autoregressive language modeling of reordered antibody sequence segments, conditioned on chain and species identifier tags. (B) Distribution of sequences in clustered OAS dataset for various species and chain types. (C) Infilling perplexity for IgLM and IgLM-S on heldout test dataset for CDR loops and random spans of 10-20 residues within sequences. (D) Effect of increased sampling temperature for full-length generation. Structures at each temperature are predicted by AlphaFold-Multimer and colored by prediction confidence (pLDDT), with a darker shade being the more confident and a lighter shade being the less confident.

FIGS. 3A-3E. Controllable antibody sequence generation. (A) Diagram of procedure for generating full-length antibody sequences given a desired species and chain type with IgLM. (B) Length of generated heavy and light with and without initial three residues provided (prompting). (C) Adherence of generated sequences to species conditioning tags. Each plot shows the species classifications of antibody sequences generated with a particular species conditioning tag (indicated above plots). Solid and dashed lines correspond to sequences generated with heavy- and light-chain conditioning, respectively. (D) Adherence of generated sequences to chain conditioning tags. Top plot shows the percentage of heavy-chain-conditioned sequences classified as heavy chains, for each species conditioning tag. Bottom plot shows the percentage of light-chain-conditioned sequences classified as lambda or kappa chains, for each species conditioning tag. (E) Effect of sampling temperature on germline identity for generated heavy and light chain sequences. As sampling temperature increases, generated sequences diverge from the closest germline V- and J-gene sequences.

FIGS. 4A-4G. Generation of infilled therapeutic antibody libraries. (A) Diagram of procedure for generating diverse antibody libraries by infilling the CDR H3 loops of therapeutic antibodies. (B) Distribution of infilled CDR H3 loop lengths for 49 therapeutic antibodies. (C) Relationship between sampling temperature (T) and nucleus probability (P) and length of infilled CDR H3 loops. (D) Infilled CDR H3 loops for trastuzumab therapeutic antibody adopt diverse lengths and conformations. Structures for infilled variants are predicted with IgFold. (E) Distribution of infilled CDR H3 loop lengths for therapeutic antibodies grouped by nearest germline gene groups. (F-G) Effect of sampling temperature (T) and nucleus probability (P) on diversity of infilled CDR H3 loops for lengths between 10 and 18 residues. Pairwise edit distance measures the minimum edits between each infilled loop to another in the same set of generated sequences (i.e., within the set of sequences produced with the same T and P parameters). For both parameters, less restrictive sampling produces greater infilled loop diversity.

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth. The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of” and “consisting essentially of,” the embodiments or elements presented herein, whether explicitly set forth or not.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, systems, and component parts, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Antibody: As used herein, the term “antibody” refers to an immunoglobulin or an antigen-binding domain thereof. The term includes but is not limited to polyclonal, monoclonal, monospecific, polyspecific, non-specific, humanized, human, canonized, canine, felinized, feline, single-chain, chimeric, synthetic, recombinant, hybrid, mutated, grafted, and in vitro generated antibodies. The antibody can include a constant region, or a portion thereof, such as the kappa, lambda, alpha, gamma, delta, epsilon and mu constant region genes. For example, heavy chain constant regions of the various isotypes can be used, including: IgG₁, IgG₂, IgG₃, IgG₄, IgM, IgA₁, IgA₂, IgD, and IgE. By way of example, the light chain constant region can be kappa or lambda. The term “monoclonal antibody” refers to an antibody that displays a single binding specificity and affinity for a particular target, e.g., epitope.

Antigen Binding Portion: As used herein, the term “antigen binding portion” refers to a portion of an antibody that specifically binds to a target antigen, e.g., a molecule in which one or more immunoglobulin chains is not full length, but which specifically binds to the target antigen. Examples of binding portions encompassed within the term “antigen-binding portion of an antibody include (i) a Fab fragment, a monovalent fragment consisting of the VLC, VHC, CL and CH1 domains: (ii) a F(ab′)2 fragment, a bivalent fragment comprising two Fab fragments linked by a disulfide bridge at the hinge region; (iii) a Fd fragment consisting of the VHC and CH1 domains; (iv) a Fv fragment consisting of the VLC and VHC domains of a single arm of an antibody, (v) a dAb fragment, which consists of a VHC domain; and (vi) an isolated complementarity determining region (CDR) having sufficient framework to specifically bind, e.g., an antigen binding portion of a variable region. An antigen binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFV). Such single chain antibodies are also encompassed within the term “antigen binding portion” of an antibody. The term “antigen binding portion” encompasses a single-domain antibody (sdAb), also known as a “nanobody” or “VHH antibody,” which is an antibody fragment consisting of a single monomeric variable antibody domain. These antibody portions are obtained using conventional techniques known to those with skill in the art, and the portions are screened for utility in the same manner as are intact antibodies.

Machine Learning Algorithm: As used herein, “machine learning algorithm,” generally refers to an algorithm, executed by computer, that automates analytical model building, e.g., for clustering, classification or pattern recognition. Machine learning algorithms may be supervised or unsupervised. Learning algorithms include, for example, artificial neural networks (e.g., back propagation networks), discriminant analyses (e.g., Bayesian classifier or Fischer analysis), support vector machines, decision trees (e.g., recursive partitioning processes such as CART—classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.”

Protein: As used herein, “protein” or “polypeptide” refers to a polymer of typically more than 50 amino acids attached to one another by a peptide bond. Examples of proteins include enzymes, hormones, antibodies, peptides, and fragments thereof
Peptide: As used herein, “peptide” refers to a sequence of 2-50 amino acids attached one to another by a peptide bond. These peptides may or may not be fragments of full proteins.
System: As used herein, “system” in the context of analytical instrumentation refers a group of objects and/or devices that form a network for performing a desired objective.

### DETAILED DESCRIPTION

Successful development of monoclonal antibodies (mAbs) for therapeutic applications is hindered by developability issues such as low solubility, low thermal stability, high aggregation, and high immunogenicity. The discovery of more developable mAb candidates relies on high-quality antibody libraries for isolating candidates with desirable properties. We present Immunoglobulin Language Model (IgLM), a deep generative language model for generating synthetic libraries by re-designing spans of antibody sequences. IgLM formulates antibody design as an autoregressive sequence generation task based on text-infilling in natural language. We trained IgLM on approximately 558M antibody heavy- and light-chain variable sequences, conditioning on each sequence's chain type and species-of-origin. We demonstrate that IgLM can be applied to generate reliable synthetic libraries, accelerating the discovery of therapeutic antibody candidates.

The present disclosure provides a method of producing a trained bidirectional generative immunoglobulin language model (IgLM) using a computer. The method includes training, by the computer, the generative IgLM using a training dataset comprising a population of reference immunoglobulin (Ig) amino acid sequence representations, wherein a given reference Ig amino acid sequence representation in the population is represented as A=(a₁, . . . , aₙ), where at is an amino acid residue at position i of the given reference immunoglobulin amino acid sequence representation, wherein a [MASK] token of length m is applied to the given reference Ig amino acid sequence representation, wherein the [MASK] token comprises a starting amino acid residue position j from [1, n−m+1] in the given reference Ig amino acid sequence representation, and wherein at least one conditioning tag is applied to the given reference Ig amino acid sequence representation, thereby producing the trained bidirectional generative IgLM. In some embodiments, the [MASK] token replaces a CDR loop span in the reference Ig amino acid sequence representation.

The present disclosure also provides a method of generating a library of synthetic antibody sequences using a computer. The method includes receiving, by the computer, at least one test immunoglobulin (Ig) amino acid sequence representation into a trained bidirectional generative immunoglobulin language model (IgLM), wherein the test Ig amino acid sequence representation is represented as A=(a₁, . . . , aₙ), where at is an amino acid residue at position i of the test Ig amino acid sequence representation, wherein a [MASK] token of length m is applied to the given test Ig amino acid sequence representation, wherein the [MASK] token comprises a starting amino acid residue position j from [1, n−m+1] in the test Ig amino acid sequence representation, and wherein at least one conditioning tag is applied to the test Ig amino acid sequence representation, wherein the bidirectional generative IgLM is trained using a training dataset comprising a population of reference immunoglobulin (Ig) amino acid sequence representations, wherein a given reference Ig amino acid sequence representation in the population is represented as A=(a₁, . . . , aₙ), where at is an amino acid residue at position i of the given reference immunoglobulin amino acid sequence representation, wherein the [MASK] token of length m is applied to the given reference Ig amino acid sequence representation, wherein the [MASK] token comprises a starting amino acid residue position j from [1, n−m+1] in the given reference Ig amino acid sequence representation, and wherein the conditioning tag is applied to the given reference Ig amino acid sequence representation, and wherein the bidirectional generative IgLM infills amino acid residues replaced by the [MASK] token applied to the given test Ig amino acid sequence representation to generate the library of synthetic antibody sequences. In some embodiments, the [MASK] token replaces a CDR loop span in the test and reference Ig amino acid sequence representations. In some embodiments, the library of synthetic antibody sequences produced by the methods disclosed herein includes variable length sequences, whereas in other embodiments, the library of synthetic antibody sequences produced by the methods disclosed herein includes sequences of uniform length. The library of synthetic antibody sequences produced by the methods disclosed herein can be of essentially any size, such as from one sequence to millions of sequences or more (e.g., about 50 sequences, about 100 sequences, about 500 sequences, about 1000 sequences, about 5000 sequences, about 10000 sequences, about 50000 sequences, about 100000 sequences, about 500000 sequences, about 1000000 sequences, about 5000000 sequences, etc.).

In some embodiments, the conditioning tag comprises a chain type, c_{c}, and/or a species-of-origin type, cₛ. In some embodiments, the chain type, c_{c}, comprises a heavy chain or a light chain. In some embodiments, the population of reference Ig amino acid sequence representations comprise monoclonal Ig amino acid sequence representations. In some embodiments, the methods disclosed herein include producing (e.g., synthesizing the amino acid sequences or nucleic acids encoding the same, expressing those nucleic acids in host organisms, or the like) a library of synthetic immunoglobulins that comprise one or more biochemical properties using the trained bidirectional generative IgLM. In some embodiments, the biochemical properties are selected from the group consisting of: a solubility level, a thermal stability level, an aggregation level, and an immunogenicity level.

In some embodiments, the [MASK] token replaces a span of amino acid residues represented as S=(aⱼ, . . . , aⱼ+m−1) in the given reference Ig amino acid sequence representation. In some embodiments, the bidirectional generative IgLM infills amino acid residues replaced by the [MASK] token. In some embodiments, the [MASK]token applied to the given reference Ig amino acid sequence representation forms a sequence representation A_{\s}=(a₁, . . . , aⱼ₋₁, [MASK], aⱼ₊ₘ, . . . , aₙ). In some embodiments, the given reference Ig amino acid sequence representation forms a sequence representation of (c_{c}, cₛ, a₁, . . . , aⱼ₋₁, [MASK] token; aⱼ₊ₘ, . . . , aₙ; [SEP] token, aⱼ, . . . , aⱼ₊ₘ₋₁, [ANS] token).

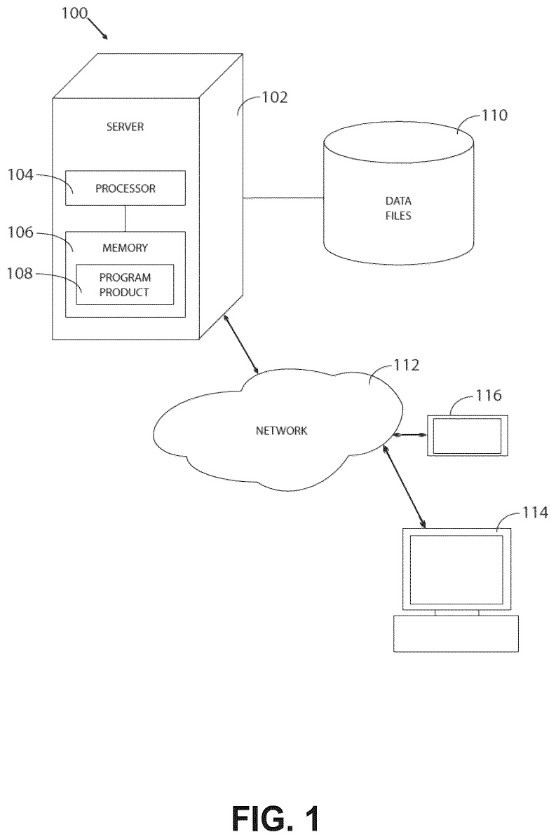

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 1 provides a schematic diagram of an exemplary system suitable for use with implementing at least aspects of the methods disclosed in this application. As shown, system 100 includes at least one controller or computer, e.g., server 102 (e.g., a search engine server), which includes processor 104 and memory, storage device, or memory component 106, and one or more other communication devices 114 and 116 (e.g., client-side computer terminals, telephones, tablets, laptops, other mobile devices, etc.) positioned remote from and in communication with the remote server 102, through electronic communication network 112, such as the Internet or other internetwork. Communication device 114 typically includes an electronic display (e.g., an internet enabled computer or the like) in communication with, e.g., server 102 computer over network 112 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 100 also includes program product 108 stored on a computer or machine readable medium, such as, for example, one or more of various types of memory, such as memory 106 of server 102, that is readable by the server 102, to facilitate, for example, a guided search application or other executable by one or more other communication devices, such as 114 (schematically shown as a desktop or personal computer). In some aspects, system 100 optionally also includes at least one database server, such as, for example, server 110 associated with an online website having data stored thereon (e.g., amino acid sequence information, etc.) searchable either directly or through search engine server 102. System 100 optionally also includes one or more other servers (e.g., comprising a trained artificial neural network (ANN)) positioned remotely from server 102, each of which are optionally associated with one or more database servers 110 located remotely or located local to each of the other servers. The other servers can beneficially provide service to geographically remote users and enhance geographically distributed operations.

As understood by those of ordinary skill in the art, memory 106 of the server 102 optionally includes volatile and/or nonvolatile memory including, for example, RAM, ROM, and magnetic or optical disks, among others. It is also understood by those of ordinary skill in the art that although illustrated as a single server, the illustrated configuration of server 102 is given only by way of example and that other types of servers or computers configured according to various other methodologies or architectures can also be used. Server 102 shown schematically in FIG. 1, represents a server or server cluster or server farm (e.g., comprising a trained ANN) and is not limited to any individual physical server. The server site may be deployed as a server farm or server cluster managed by a server hosting provider. The number of servers and their architecture and configuration may be increased based on usage, demand and capacity requirements for the system 100. As also understood by those of ordinary skill in the art, other user communication devices 114 and 116 in these aspects, for example, can be a laptop, desktop, tablet, personal digital assistant (PDA), cell phone, server, or other types of computers. As known and understood by those of ordinary skill in the art, network 112 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 108 is optionally in the form of microcode, programs, cloud computing format, routines, and/or symbolic languages that provide one or more sets of ordered operations that control the functioning of the hardware and direct its operation. Program product 108, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 108 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 108 is optionally copied from the computer-readable medium to a hard disk or a similar intermediate storage medium. When program product 108, or portions thereof, are to be run, it is optionally loaded from their distribution medium, their intermediate storage medium, or the like into the execution memory of one or more computers, configuring the computer(s) to act in accordance with the functionality or method of various aspects. All such operations are well known to those of ordinary skill in the art of, for example, computer systems.

To further illustrate, in certain aspects, this disclosure provides systems that include one or more processors, and one or more memory components in communication with the processor. The memory component typically includes one or more instructions that, when executed, cause the processor to provide information that causes a library of synthetic antibody sequences and/or the like to be displayed (e.g., via communication device 114 or the like) and/or receive information from other system components and/or from a system user (e.g., via communication device 114 or the like).

In some aspects, program product 108 includes non-transitory computer-executable instructions which, when executed by electronic processor 104 perform at least: receiving at least one test immunoglobulin (Ig) amino acid sequence representation into a trained bidirectional generative immunoglobulin language model (IgLM), wherein the test Ig amino acid sequence representation is represented as A=(a₁, . . . , aₙ), where at is an amino acid residue at position i of the test Ig amino acid sequence representation, wherein a [MASK] token of length m is applied to the given test Ig amino acid sequence representation, wherein the [MASK] token comprises a starting amino acid residue position j from [1, n−m+1] in the test Ig amino acid sequence representation, and wherein at least one conditioning tag is applied to the test Ig amino acid sequence representation, wherein the bidirectional generative IgLM is trained using a training dataset comprising a population of reference immunoglobulin (Ig) amino acid sequence representations, wherein a given reference Ig amino acid sequence representation in the population is represented as A=(a₁, . . . , aₙ), where at is an amino acid residue at position i of the given reference immunoglobulin amino acid sequence representation, wherein the [MASK] token of length m is applied to the given reference Ig amino acid sequence representation, wherein the [MASK] token comprises a starting amino acid residue position j from [1, n−m+1] in the given reference Ig amino acid sequence representation, and wherein the conditioning tag is applied to the given reference Ig amino acid sequence representation, and wherein the bidirectional generative IgLM infills amino acid residues replaced by the [MASK] token applied to the given test Ig amino acid sequence representation to generate a library of synthetic antibody sequences.

### EXAMPLE: Generative Language Modeling for Antibody Design

### 1. Introduction

We present Immunoglobulin Language Model (IgLM), which leverages bidirectional context for designing antibody sequence spans of varying lengths while training on a large-scale natural antibody dataset. We show that IgLM can generate full-length antibody sequences conditioned on chain type and species-of-origin. Furthermore, IgLM can diversify loops on an antibody to generate high-quality libraries that display favorable biophysical properties while resembling human antibodies. IgLM should be a powerful tool for antibody discovery and optimization.

### 2. Results

Immunoglobulin language model. Our method for antibody sequence generation, IgLM, is trained on 558 million natural antibody sequences for both targeted infilling of residue spans, as well as full-length sequence generation. IgLM generates sequences conditioned on the species-of-interest and chain type (heavy or light), enabling controllable generation of antibody sequences.

Infilling language model. Design of antibody libraries typically focuses on diversification of the CDR loop sequences, in order to facilitate binding to a diverse set of antigens. Existing approaches to protein sequence generation (including antibodies) typically adopt left-to-right decoding strategies. While these models have proven effective for generation of diverse and functional sequences, they are ill-equipped to re-design specific segments of interest within proteins. To address this limitation, we developed IgLM, an infilling language model for immunoglobulin sequences. IgLM utilizes a standard left-to-right decoder-only transformer architecture (GPT-2), but is trained for infilling through rearrangement of sequences. Specifically, we adopt the infilling language model formulation from natural language processing, wherein arbitrary-length sequence segments (spans) are masked during training and appended to the end of the sequence. By training on these rearranged sequences, models learn to predict the masked spans conditioned on the surrounding sequence context.

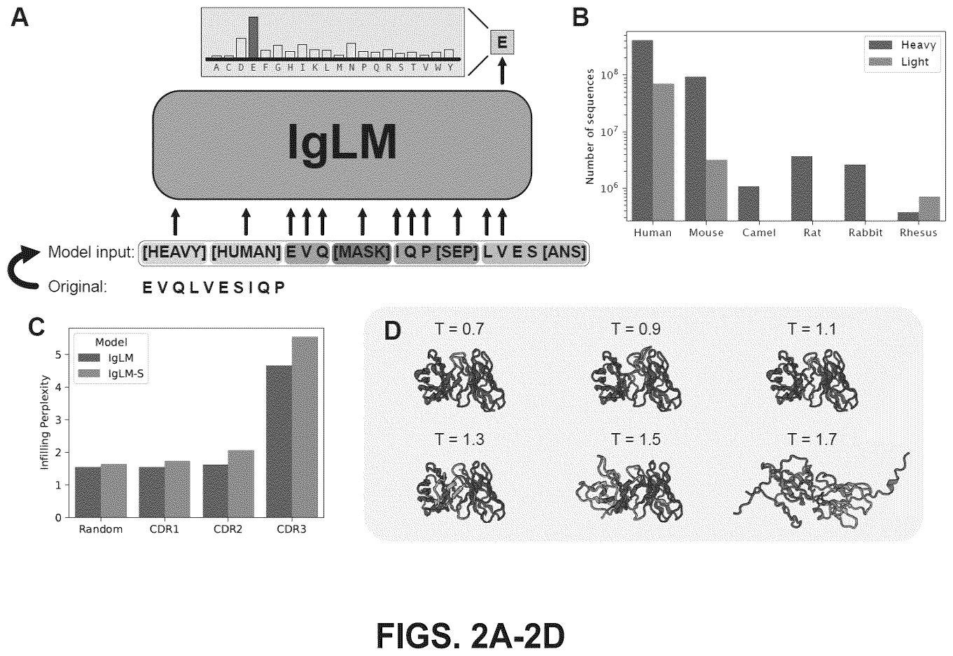

To train IgLM, we collected antibody sequences from the Observed Antibody Space (OAS). The OAS database contains natural antibody sequences from six species: human, mouse, rat, rabbit, rhesus, and camel. To investigate the impacts of model capacity, we trained two versions of the model: IgLM and IgLM-S, with 13M and 1.4M trainable parameters, respectively. Both IgLM models were trained on a set of 558M non-redundant sequences, clustered at 95% sequence identity. During training, we randomly masked spans of ten to twenty residues within the antibody sequence to enable diversification of arbitrary spans during inference. Additionally, we condition sequences on the chain type (heavy or light) and species-of-origin. Providing this context enables controllable generation of species-specific antibody sequences. An example of training data construction is illustrated in FIG. 2A. Unless otherwise specified, we use the larger IgLM model for all experiments.

IgLM generates foldable antibody sequences. As an initial validation of the antibody sequence generation capabilities of IgLM, we conducted a small scale investigation of full-length generation. Specifically, we investigated the impacts of sampling temperature for tuning the diversity of generated sequences. Sampling temperature values above one effectively flatten the amino acid distribution at each step of generation, resulting in more diverse sequences, while temperature below one sharpens the distribution at each position, resembling a greedy decoding strategy. We generated a set of full-length sequences at temperatures ranging from 0.7 to 1.7, providing the model with human heavy and human light conditioning tags. Because IgLM was trained for sequence infilling, generated sequences contain discontinuous segments of sequence segments, which we simply reordered to produce full-length antibodies. Generated heavy and light chain sequences were paired according to sampling temperature and their structures were predicted using AlphaFold-Multimer. In general, IgLM generates sequences with correspondingly confident predicted structures at lower temperatures (up to 1.3), before beginning to degrade in quality at higher temperatures (FIG. 2D).

Language modeling evaluation. We evaluated IgLM as a language model by computing the per-token perplexity for infilled spans within an antibody, which we term the infilling perplexity. Computing the infilling perplexity is equivalent to taking the per-token perplexity after the [SEP] token. We compared the infilling perplexity of IgLM and IgLM-S on a heldout test dataset of 30M sequences (FIG. 2C). Results are tabulated by CDR loop, as well as for spans selected randomly within the antibody sequence. As expected, we observe greater perplexity for the CDR loops than the randomly chosen spans, which include the highly conserved framework regions. The CDR3 loop, which is the longest and most diverse, has the highest infilling perplexity. When we compare IgLM and IgLM-S, we observe that IgLM has a lower infilling perplexity for all CDR loops, indicating that the larger IgLM model (with ten times more parameters) is better at modeling the diversity of antibody sequences.

The diversity of antibody sequences varies by species and chain type. For example, heavy chains introduce additional diversity into their CDR3 loops via D-genes, while some species (e.g., camels) tend to have longer loops. To investigate how these differences impact the performance of IgLM in different settings, we also tabulated the heldout set infilling perplexity by species and chain type. In general, both IgLM models achieve low infilling perplexity for random spans across all species. For CDR1 and CDR2 loop infilling, perplexity values are typically lower for human and mouse antibodies, which are disproportionately represented in the OAS database. In general, both models still perform better on these loops than the more challenging CDR3 loops, regardless of species. One exception is for rhesus CDR2 loops, on which IgLM-S performs considerably worse than the larger IgLM model. This appears to be due to poor fitting of rhesus CDR L2 loops, as reflected in the similarity high infilling average perplexity observed when tabulated by chain type. The highest infilling perplexity is observed for camel CDR3 loops, which tend to be longer than other species. Across all species and chain types, the larger IgLM model achieves lower infilling perplexity than IgLM-S, suggesting that further increasing model capacity would yield additional improvements.

Controllable generation of antibody sequences. Having demonstrated that IgLM can generate well-formed full-lengths sequences, we next considered the controllability of IgLM for generating antibody sequences with specific traits. Controllable generation utilizes conditioning tags to provide the model with additional context about the expected sequence. We generated a set of 220K sequence to evaluate the behavior of IgLM given particular species-of-origin and chain type tags.

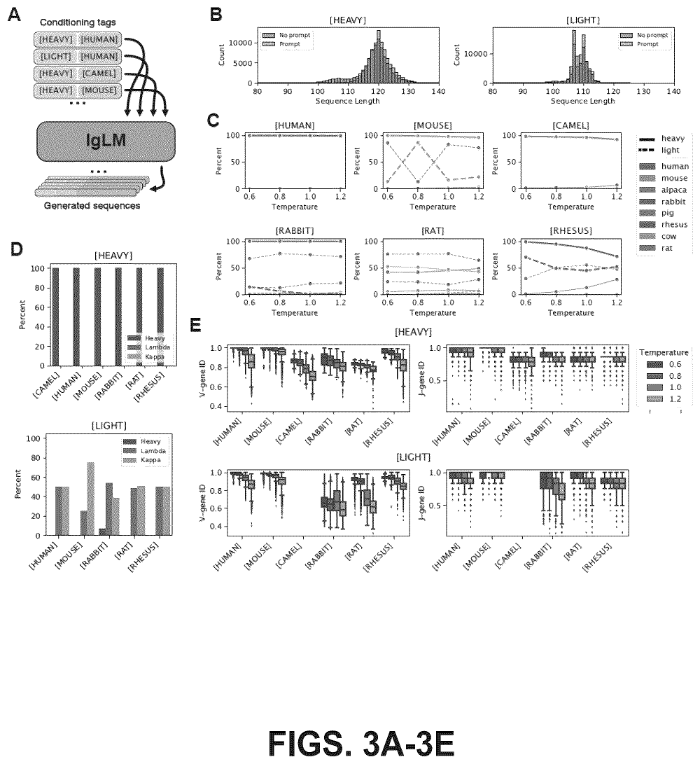

Generating species- and chain-controlled sequences. To evaluate the controllability of IgIM, we generated a set of 220K full-length sequences utilizing all viable combinations of conditioning tags, as well as a range of sampling temperatures (FIG. 3A). For every species (except camel), we combined sampled with both heavy and light conditioning tags. For camel sequence generation, we only sampled heavy chains, as they do not produce light chains. To produce a diverse set of sequences for analysis, we sampled using a range of temperatures (T ∈{0.6, 0.8, 1.0, 1.2}). Sampling under these conditions resulted in a diverse set of antibody sequences. However, we observed that the sequences frequently featured N-terminal truncations, a common occurrence in the OAS database used for training. For heavy chains, these N-terminal deletions appeared as a left-shoulder in the sequence length distribution (FIG. 3B, left) with lengths ranging from 100 to 110 residues. For light chains, we observed a population of truncated chains with lengths between 98 and 102 residues (FIG. 3B, right). To address truncation in generated sequences, we utilized a prompting strategy, wherein we initialize each sequence with a three-residue motif corresponding to the species and chain type tags. For both heavy and light chains, prompting with initial reisdues significantly reduced the population of truncated sequences (FIG. 3B). For the following analysis, we consider only sequences generated with prompting.

Adherence to conditioning tags. To evaluate the effectiveness of controllable generation, we considered the agreement between the provided conditioning tags and the sequences produced by IgLM. For each generated sequence, we classified the species (according to V-gene identity) and chain type using ANARCI. We note that the species classes provided by ANARCI diverge in some cases from those provided by the OAS database, but there was a suitable corresponding class for each conditioning token. In FIG. 3C, we show the makeup of sequences for each species conditioning tag, according to sampling temperature. In each plot, the percentage of heavy and light chain sequences classified as each species are indicated by solid and dashed lines, respectively. For most species (human, mouse, camel, rabbit, rhesus), IgLM is able to successfully generate heavy chain sequences at every temperature. The exception to this trend is rat sequences, for which we were unable to produce any sequences that ANARCI classified as belonging to the intended species.

The ability to generate sequences is not directly explained by prevalence in the training dataset, as the model is trained on an order of magnitude more rat heavy chain sequences than rhesus. IgLM is generally less effective at generating light chain sequences for most species. With the exception of human light chains, all species have a large proportion of sequences classified as belonging to an unintended species (typically human). For mouse and rhesus light chains, IgLM generates the correct species in 34.89% and 88.14% of cases, respectively. For rabbit and rat light chains, IgLM was not exposed to any examples during training. Interestingly, despite having seen no such sequences during training, IgLM is capable of generating sequences classified by ANARCI as rabbit light chains for 6.89% of samples. The majority of these sequences are cases where the model has instead generated a rabbit heavy chain. However, for 35 of these 1120 cases, IgLM has produced rabbit light chain sequences. This suggests that IgLM has learned some generalizable notion of rabbit antibodies, which is composable with its conditioning for producing light chains.

We next evaluated the adherence of IgLM-generated sequences to chain type conditioning tags. In FIG. 3D, we show the percentage of sequences classified by ANARCI as heavy or light for each conditioning tag. Light chains are further divided into lambda and kappa. When conditioned towards heavy chain generation, IgLM effectively produces heavy chains for all species. For light chains, we observe a similar trend, with IgLM producing predominantly light chain sequences for all species. Only for rabbit sequences do we observe a population of heavy chains when conditioning for light chains. As noted above, these are cases where IgLM has instead produced a rabbit heavy chain. When generating light chain sequences, we provide initial residues characteristic of both lambda and kappa chains in equal proportion. For most species, this results in roughly equal fractions of lambda and kappa chains. However, for mouse sequences, we observe a bias towards kappa chains. This may be reflective of an imbalance in the training dataset and could likely be resolved through improved prompting strategies.

Sampling temperature controls mutational load. Increasing sampling temperature has the effect of flattening the probability distribution at each position during sampling, resulting in a greater diversity of sequences. We evaluated the effect of sampling temperature on the diversity of generated sequences by measuring the fractional identity to the closest germline sequences using ANARCI. In FIG. 3E, we show the germline identity for V- and J-genes for each species and chain type. At the lowest sampling temperature (T=0.6), IgLM frequently recapitulates germline sequences in their entirety for some species (human, mouse, rhesus). As temperature increases, sequences for every species begin to diverge from germline, effectively acquiring mutations. Interestingly, J-gene sequences typically acquire fewer mutations than V-genes for both heavy and light chains. This is likely a reflection of the concentration of CDR loops within the V-gene (CDR1 and CDR2). Only a portion of the CDR3 loop is contributed by the J-gene, with the remaining sequence being conserved framework residues.

Therapeutic antibody diversification. Diversification of antibody CDR loops is a common strategy for antibody discovery or optimization campaigns. Through infilling, IgLM is capable of replacing spans of amino acids within antibody sequences, conditioned on the surrounding context. To demonstrate this functionality, we generated infilled libraries for a set of therapeutic antibodies and evaluated several therapeutically relevant properties.

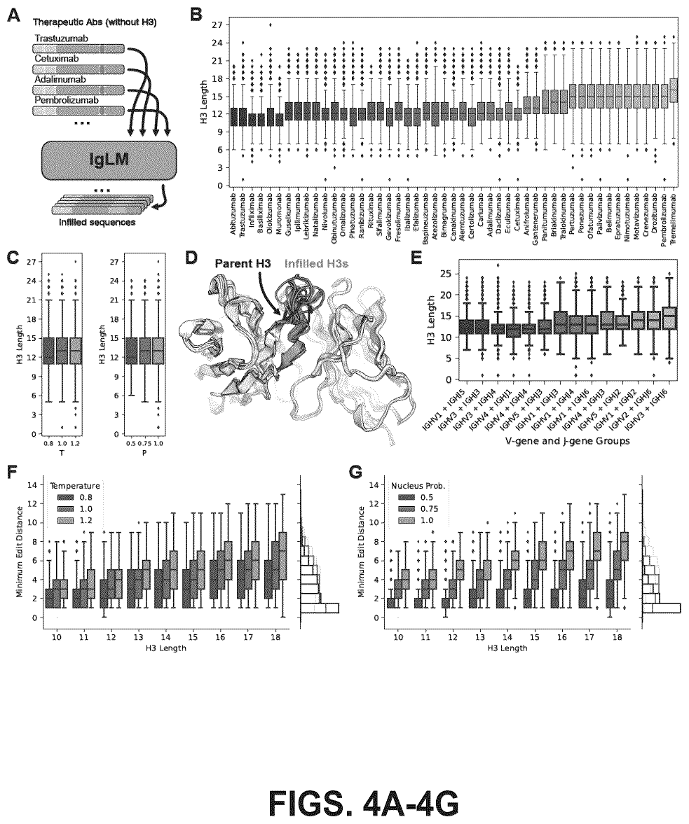

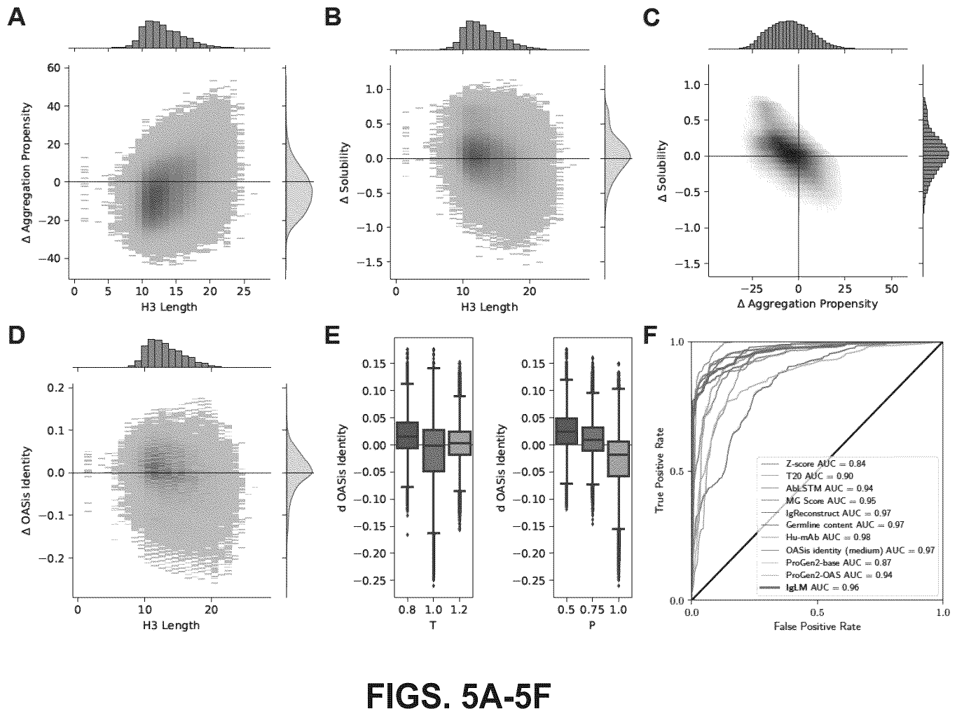

Infilled libraries for therapeutic antibodies. To evaluate the utility of infilling with IgLM for diversifying antibody sequences, we created infilled libraries for 49 therapeutic antibodies from Thera-SAbDab. For each antibody, we removed the CDR H3 loop and generated a library of infilled sequences using IgLM (FIG. 4A). To produce diverse sequences, we used a combination of sampling temperatures (T ∈{0.8, 1.0, 1.2}) and nucleus sampling probabilities (P ∈ {0.5, 0.75, 1.0}). Nucleus sampling effectively clips the probability distribution at each position during sampling, such that only the most probable amino acids (summing to P) are considered. For each of the 49 therapeutic antibodies, we generated one thousand infilled sequences for each combination of T and P, totaling nine thousand variants per parent antibody. In FIG. 4D, we show predicted structures (using IgFold) for a subset of ten infilled loops derived from the trastuzumab antibody. The infilled loops vary in length and adopt distinct structural conformations. Across the infilled libraries, we see a variety of infilled CDR H3 loop lengths, dependent on the parent antibody's surrounding sequence context (FIG. 4B). The median length of infilled loops across antibodies ranges from 11 to 16 residues. Interestingly, we observe little impact on the length of infilled loops when varying the sampling temperature and nucleus probabilities (FIG. 4C).

The distributions of infilled loop lengths vary considerably over the 49 therapeutic antibodies here. Because IgLM is trained on natural antibody sequences, we hypothesized that the model may be performing a sort of germline matching, wherein sequences with similar V- and J-genes lead to similar distributions of loop lengths. To test this, we identified the closest germline genes for each antibody with ANARCI. We then group parent antibodies according to common V- and J-gene groups and compared the distributions of infilled loop lengths for each group (FIG. 4E). While there may be some tendency for similar V- and J-genes to lead to similar distributions of infilled loop lengths, we observe considerable variation. This suggests that IgLM is not purely performing germline matching, but rather is considering other properties of the parent antibody.

FIGD. 5A-5F. Therapeutic properties of infilled antibody libraries. (A) Change in predicted aggregation propensity of infilled sequences relative to their parent antibodies. Infilled sequences typically display reduced aggregation propensity (negative is improved), particularly for shorter loops. (B) Change in predicted solubility of infilled sequences relative to their parent antibodies. Infilled sequences typically display increased solubility (positive is improved). (C) Relationship between predicted changes in aggregation propensity and solubility for infilled sequence libraries. (D) Change in humanness of infilled sequences relative to their parent antibodies. Humanness is calculated as the OASis identity of the heavy chain sequence, with positive larger values being more humanlike. (E) Relationship between sampling temperature (T) and nucleus probability (P) and change in human-likeness (OASis identity) of infilled heavy chains relative to their parent sequences. (F) Receiver operating characteristic (ROC) curves for human sequence classification methods. The area under the curve (AUC) is shown for each method.

Infilling generates diverse loop sequences. Diverse loop libraries are essential for discovering or optimizing sequences against an antigen target. To evaluate the diversity of infilled loops produced by IgLM, we measured the pairwise edit distance between each loop sequence and its closest neighbor amongst the sequences generated with the same sampling parameters. We then compared the diversity of sequences according to loop length and choice of sampling parameters (FIGS. 4F-G). Generally, we observe that generated loops are more diverse at longer lengths, as expected given the increased combinatorial complexity available as more residues are added. Increasing both sampling temperature and nucleus probability results in a greater diversity of sequences. However, these parameters affect the relationship between length and diversity in distinct ways. For a given loop length, increasing temperature produces more variance in the pairwise edit distance, while increases to nucleus probability provides a more consistent increase in diversity across loop lengths. Indeed, the marginal distribution of pairwise edit distance as nucleus probability is increased produces a much larger shift (FIG. 4G, marginal) than that of temperature (FIG. 4F, marginal). In practice, a combination of sampling parameters may be suitable for producing a balance of high-likelihood (low temperature and low nucleus probability) and diverse sequences.

Infilled loops display improved developability. Developability encompasses a set physiochemical properties—including aggregation propensity and solubility—that are critical for the success of a therapeutic antibody. Libraries for antibody discovery or optimization that are enriched for sequences with improved developability can alleviate the need for time-consuming post hoc engineering. To evaluate the developability of sequences produced by IgLM, we used high-throughput computational tools to calculate the aggregation propensity (SAP score) and solubility (CamSol Intrinsic) of the infilled therapeutic libraries. As a precursor to calculation of aggregation propensity, we used IgFold to predict the structures of the infilled antibodies (including the unchanged light chains). We then compared the aggregation propensities and solubility values of the infilled sequences to those of the parent antibodies. For aggregation propensity, we observed a significant improvement (negative is better) by infilled sequences over the parent antibodies (FIG. 5A). For solubility, we observed a significant improvement for infilled sequences over the parent antibodies (FIG. 5B). Similarly for solubility, infilled sequences tend to be more soluble than their parent antibodies (FIG. 5B). In both cases, the largest improvements tend to correspond to the shorter loops. Further, we observe a positive correlation between improvements to aggregation propensity and solubility (FIG. 5C). These results suggest that infilling can be used to generate libraries enriched for sequences with improved developability.

We next investigated whether choice of sampling parameters affects the developability of infilled sequences. When we compared the aggregation propensity and solubility of infilled sequences according to the sampling temperature and nucleus sampling probability, we found no significant differences. This is likely explained by the relative consistency of infilled loop lengths across sampling temperatures (FIG. 4C). These results suggests that developability should not be a concern when determining the appropriate diversity of a generated library.

Infilled loops are more human-like. Therapeutic antibodies must be human-like to avoid provoking an immune response and to be safe for use in humans. To evaluate the human-likeness of infilled sequences, we calculated the OASis identity (at medium stringency). OASis divides an antibody sequence into a set of 9-mers and calculates the fraction that have been observed in human repertoires. Thus, higher OASis identity indicates a sequence that is more similar to those produced by humans. When compared to their respective parent antibodies, sequences infilled by IgLM were typically more human-like (FIG. 5D). This is expected, given that IgLM is trained on natural human antibodies. We also investigated the impact of sampling parameters on the human-likeness of infilled sequences. For both sampling temperature and nucleus probability, we find that less restrictive sampling tends to produce less human-like sequences (FIG. 5E). For practical purposes, this suggests that sampling with lower temperature and nucleus probability may be more suitable if immunogenicity is a concern.

Sequence likelihood is an effective predictor of humanness. Likelihoods from autoregressive language models trained on proteins have been shown to be effective zero-shot predictors of protein fitness. Antibody-specific language models in particular have been used to measure the “naturalness” of designed sequences, a measure related to humanness. To evaluate the effectiveness of IgLM for distinguishing human from non-human antibodies, we utilized the model's likelihood to classify sequences from the IMGT mAb DB. Sequences in this set span a variety of species (human and mouse) and engineering strategies (e.g., humanized, chimeric, felinized). We considered all sequences not specifically labeled as human to be non-human, and calculated a likelihood (conditioned on human species) for each. All sequences had both a heavy and light chain, which we calculated likelihoods for separately then multiplied.

We compared the performance of IgLM to that of a number of other methods previously benchmarked by Prihoda et al (A platform for antibody design, humanization and humanness evaluation based on natural antibody repertoires and deep learning. bioRxiv, 2021) using a receiver operating characteristic (ROC) curve (FIG. 5F). The results here for alternative methods are adapted from those presented by Prihoda et al (Id.), but with several redundant entries removed to avoid double-counting. We additionally evaluated model likelihoods from ProGen2-base and ProGen2-OAS, which are similar models to IgLM, but contain significantly more parameters (764M). ProGen2-base is trained on a diverse set of protein sequences, while ProGen2-OAS is trained on a similar dataset to IgLM (OAS clustered at 85% sequence identity). We find that IgLM is competitive with state-of-the-art methods designed for human sequence classification, though not the best. Interestingly, IgLM outperforms ProGen2-OAS (ROC AUC of 0.96 for IgLM vs. 0.94 for ProGen2-OAS), despite having significantly fewer parameters (13M vs. 764M). This may be a product of different strategies for constructing training datasets from OAS. By filtering at a less stringent 95% sequence identity, IgLM is likely exposed to a greater proportion of human antibody sequences, which dominate the OAS database. These distinctions highlight the importance of aligning training datasets with the intended application, and suggest that training on only human sequences may further improve performance for human sequence classification.

### 3. Discussion

Antibody libraries are a powerful tool for discovery and optimization of therapeutics. However, they are hindered by large fractions of non-viable sequences, poor developability, and immmunogenic risks. Generative language models offer a promising alternative to overcome these challenges, through on-demand generation of high-quality sequences. However, previous work has focused entirely on contiguous sequence decoding (N-to-C or C-to-N). While useful, such models are not well-suited for generating antibody libraries, which vary in well-defined regions within the sequence, and for which changes may be undesirable in other positions. In this work, we presented IgLM: an antibody-specific language model for generation of full-length sequences and infilling of targeted residue spans. IgLM is trained for sequence infilling on 558M natural antibody sequences from six species. During training, we provide the model with conditioning tags that indicate the antibody's chain type and species-of-origin, enabling controllable generation of desired types of sequences.

Concurrent work on autoregressive language models for antibody sequence generation have been trained on similar sets of natural antibody sequences and explored larger model sizes. However, models like ProGen2-OAS are limited in utility for antibody generation and design, as they are difficult to guide towards generation of specific types of sequences (e.g., species or chain types). Both this work and the ProGen2-OAS paper have utilized prompting strategies to guide model generation towards full-length sequences. While these strategies may help in some cases (particularly to overcome dataset limitations), significantly more residues may need to be provided to guide the model towards a specific sequence type (e.g., human vs rhesus heavy chain). In contrast, by including conditioning information for species and chain type in the model's training, IgLM is able to generate sequences of the desired type without additional prompting. Still, as shown in this work, increasing the capacity of models like IgLM may lead to better performance for sequence infilling (lower perplexity) and scoring (better likelihood estimation) and is a promising direction for future work.

Among IgLM's innovations is the ability to generate infilled residue spans at specified positions within the antibody sequence. In contrast to traditional generative language models that only consider preceding the residues, this enables IgLM to generate within the full context of region to be infilled. We demonstrate the utility of infilling by generating libraries for 49 therapeutic antibodies. We found that IgLM was capable of generating diverse CDR H3 loop sequences, and that diversity was largely tunable by choice of sampling parameters. Further, the infilled libraries possessed desirable developability traits (aggregation propensity, solubility) while being more human-like on average than their parent sequences. Notably, IgLM achieves these improvements over antibodies that are already highly optimized, as all of the parent sequences have been engineered for mass-production and use in humans. Although we focused on antibody loop infilling in this work, similar strategies may be useful for proteins generally. For example, a universal protein sequence infilling model may be applicable to redesign of contiguous protein active sites, or for generating linkers between disparate domains for protein engineering.

### 4. Methods

Infilling formulation. Designing spans of amino acids within an antibody sequence can be formulated as an infilling task, similar to text-infilling in natural language. We denote an antibody sequence A=(a₁, . . . aₙ), where aᵢ represents the amino acid at position i of the antibody sequence. To design a span of length m starting at position j along the sequence, we first replace the span of amino acids S=(aⱼ, . . . aⱼ₊ₘ₋₁) with a single [MASK] token to form a sequence A_{\S} S=(a₁, . . . aⱼ₋₁, [MASK], aⱼ₊ₘ, . . . aₙ). To generate reasonable variable-length spans to replace S given A_{\S}, we seek to learn a distribution p(S|A_{\S}).

We draw inspiration from the Infilling by Language Modeling (ILM) framework proposed for natural language infilling to learn p(S|A_{\S}). For assembling the model input, we first choose a span S and concatenate A_{\S}, [SEP],S, and [ANS]. We additionally prepend conditioning tags c_{C} and c_{S} to specific the chain type (heavy or light) and species-of-origin (e.g., human, mouse, etc.) of the antibody sequence. The fully formed sequence of tokens X for IgLM is:

$$X = ( {c}_{c} , {c}_{s} , {a}_{1} , … {a}_{j - 1} , [ MASK ] , {a}_{j + m} , … {a}_{n} , [ SEP ] , {a}_{j} , … {a}_{j + m - 1} , [ ANS ] ) \qquad [ 1 ]$$

We then train a generative model with parameters θ to maximize p(X|θ), which can be decomposed into a product of conditional probabilities:

$${max}_{θ} p ( X \vert θ ) = {max}_{θ} {\prod }_{i} p ( {X}_{i} \vert {X}_{< i} , θ ) \qquad [ 2 ]$$

Model implementation. The IgLM model uses a modified version of the GPT-2 Transformer decoder architecture as implemented in the HuggingFace Transformers library. We trained two models, IgLM and IgLM-S, for sequence infilling. Hyperparameter details are provided in Table 1.

TABLE 1
IgLM model hyperparameters

| | IgLM | IgLM-S |
|---|---|---|
| Number of layers | 4 | 3 |
| Embedding dimension | 512 | 192 |
| Hidden dimension | 512 | 192 |
| Attention heads | 8 | 6 |
| Feed-forward dimension | 2048 | 768 |
| Total parameters | 12,888,576 | 1,439,232 |

Antibody sequence dataset. To train IgLM, we collected unpaired antibody sequences from the Observed Antibody Space (OAS). OAS is a curated set of over one billion unique antibody sequences compiled from over eighty immune repertoire sequencing studies. After removing sequences indicated to have potential sequencing errors, we were left with 809M unique antibody sequences. We then clustered these sequences using LinClust at 95% sequence identity, leaving 588M non-redundant sequences. The distribution of sequences corresponding to each species and chain type are documented in FIG. 2B. We note that the dataset is heavily skewed towards human antibodies, particularly heavy chains, which make up 70% of all sequences. We held out 5% of sequences as a test set to evaluate model performance. Of the remaining sequences, we used 558M sequences for training and 1 M for validation.

Model training. During training, for each sequence A=(a₁, . . . , aₙ) we chose a mask length m uniformly at random from [10, 20] and a starting position j uniformly at random from [1, n−m+1]. We prepended two conditioning tags c_{C} and c_{S} denoting the chain type and species-of-origin of each sequence as annotated in the OAS database. Models were trained with a batch size of 512 and 2 gradient accumulation steps using DeepSpeed. Training took approximately 3 days when distributed across 4 NVIDIA A100 GPUs.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, cranial implant devices, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

The invention claimed is:

1. A method of generating a library of synthetic antibody sequences using a computer, the method comprising receiving, by the computer, at least one test immunoglobulin (Ig) amino acid sequence representation into a trained bidirectional generative immunoglobulin language model (IgLM),
wherein the test Ig amino acid sequence representation is represented as A=(a₁, . . . , aₙ),
where a₁ is an amino acid residue at position i of the test Ig amino acid sequence representation, wherein a [MASK] token of length m is applied to the given test Ig amino acid sequence representation, wherein the [MASK] token comprises a starting amino acid residue position j from [1, n−m+1] in the test Ig amino acid sequence representation, and wherein at least one conditioning tag is applied to the test Ig amino acid sequence representation,
wherein the bidirectional generative IgLM is trained using a training dataset comprising a population of reference immunoglobulin (Ig) amino acid sequence representations, wherein a given reference Ig amino acid sequence representation in the population is represented as A=(a₁, . . . , aₙ), where a_{I} is an amino acid residue at position i of the given reference immunoglobulin amino acid sequence representation, wherein the [MASK] token of length m is applied to the given reference Ig amino acid sequence representation, wherein the [MASK] token comprises a starting amino acid residue position j from [1, n−m+1] in the given reference Ig amino acid sequence representation, and wherein the conditioning tag is applied to the given reference Ig amino acid sequence representation, and
wherein the bidirectional generative IgLM infills amino acid residues replaced by the [MASK] token applied to the given test Ig amino acid sequence representation to generate the library of synthetic antibody sequences.

2. The method of claim 1, wherein the conditioning tag comprises a chain type, c_{c}, and/or a species-of-origin type, cₛ.

3. (canceled)

4. The method of claim 1, wherein the population of reference Ig amino acid sequence representations comprise monoclonal Ig amino acid sequence representations.

5. The method of claim 1, comprising producing a library of synthetic immunoglobulins that comprise one or more biochemical properties using the trained bidirectional generative IgLM.

6. The method of claim 5, wherein the biochemical properties are selected from the group consisting of: a solubility level, a thermal stability level, an aggregation level, and an immunogenicity level.

7. The method of claim 1, wherein the [MASK] token replaces a span of amino acid residues represented as S=(aⱼ, . . . , aⱼ₊ₘ₋₁) in the given reference Ig amino acid sequence representation.

8. The method of claim 1, wherein the bidirectional generative IgLM infills amino acid residues replaced by the [MASK] token.

9. The method of claim 1, wherein the [MASK] token applied to the given reference Ig amino acid sequence representation forms a sequence representation A_{\S}=(a₁, . . . , aⱼ₋₁, [MASK], aⱼ₊ₘ, . . . , aₙ).

10. The method of claim 1, wherein the given reference Ig amino acid sequence representation forms a sequence representation of (c_{c}, cₛ, a₁, . . . , aⱼ₋₁, [MASK] token; aⱼ₊ₘ, . . . , aₙ; [SEP] token, aⱼ, . . . , aⱼ₊ₘ₋₁, [ANS] token).

11. The trained bidirectional generative IgLM produced by the method of claim 1.

12. The library of synthetic antibody sequences produced by the method of claim 1.

13-27. (canceled)

28. A system comprising at least one controller that comprises, or is capable of accessing, computer readable media comprising non-transitory computer executable instructions which, when executed by at least one electronic processor, perform at least: receiving at least one test immunoglobulin (Ig) amino acid sequence representation into a trained bidirectional generative immunoglobulin language model (IgLM), wherein the test Ig amino acid sequence representation is represented as A (a₁, . . . , aₙ), where a_{I} is an amino acid residue at position i of the test Ig amino acid sequence representation, wherein a [MASK] token of length m is applied to the given test Ig amino acid sequence representation, wherein the [MASK] token comprises a starting amino acid residue position j from [1, n−m+1] in the test Ig amino acid sequence representation, and wherein at least one conditioning tag is applied to the test Ig amino acid sequence representation, wherein the bidirectional generative IgLM is trained using a training dataset comprising a population of reference immunoglobulin (Ig) amino acid sequence representations, wherein a given reference Ig amino acid sequence representation in the population is represented as A (a₁, . . . , aₙ), where aᵢ is an amino acid residue at position i of the given reference immunoglobulin amino acid sequence representation, wherein the [MASK] token of length m is applied to the given reference Ig amino acid sequence representation, wherein the [MASK] token comprises a starting amino acid residue position j from [1, n−m+1] in the given reference Ig amino acid sequence representation, and wherein the conditioning tag is applied to the given reference Ig amino acid sequence representation, and wherein the bidirectional generative IgLM infills amino acid residues replaced by the [MASK] token applied to the given test Ig amino acid sequence representation to generate a library of synthetic antibody sequences.

29. (canceled)

30. (canceled)

31. The system of claim 28, wherein the population of reference Ig amino acid sequence representations comprise monoclonal Ig amino acid sequence representations.

32. The system of claim 28, wherein the non-transitory computer executable instructions which, when executed by the electronic processor, further perform at least: producing a library of synthetic immunoglobulins that comprise one or more biochemical properties using the trained bidirectional generative IgLM, wherein the biochemical properties are selected from the group consisting of: a solubility level, a thermal stability level, an aggregation level, and an immunogenicity level.

33. The system of claim 28, wherein the [MASK] token replaces a span of amino acid residues represented as S=(aⱼ, . . . , aⱼ₊ₘ₋₁) in the given reference Ig amino acid sequence representation.

34. The system of claim 28, wherein the bidirectional generative IgLM infills amino acid residues replaced by the [MASK] token.

35. The system of claim 28, wherein the [MASK] token applied to the given reference Ig amino acid sequence representation forms a sequence representation A_{\S}=(a₁, . . . , aⱼ₋₁, [MASK], aⱼ₊ₘ, . . . , aₙ).

36. The system of claim 28, wherein the given reference Ig amino acid sequence representation forms a sequence representation of (c_{c}, cₛ, a₁, . . . , aⱼ₋₁, [MASK] token; aⱼ₊ₘ, . . . , aₙ; [SEP] token, aⱼ, . . . , aⱼ₊ₘ₋₁, [ANS] token).

37. The system of claim 28, wherein the 2D unmodeled bias is a function of radial detector bin and projection angle.

38.-40. (canceled)

41. A method of generating a library of synthetic peptide or protein sequences using a computer, the method comprising receiving, by the computer, at least one test amino acid sequence representation into a trained model,
wherein the test amino acid sequence representation is represented as A=(a₁, . . . , aₙ), where aᵢ is an amino acid residue at position i of the test amino acid sequence representation, wherein a [MASK] token of length m is applied to the given test amino acid sequence representation, wherein the [MASK] token comprises a starting amino acid residue position j from [1, n−m+1] in the test amino acid sequence representation, and wherein at least one conditioning tag is applied to the test amino acid sequence representation,
wherein the model is trained using a training dataset comprising a population of reference amino acid sequence representations, wherein a given reference amino acid sequence representation in the population is represented as A (a₁, . . . , aₙ), where aᵢ is an amino acid residue at position i of the given reference amino acid sequence representation, wherein the [MASK] token of length m is applied to the given reference amino acid sequence representation, wherein the [MASK] token comprises a starting amino acid residue position j from [1, n−m+1] in the given reference amino acid sequence representation, and wherein the conditioning tag is applied to the given reference amino acid sequence representation, and
wherein the model infills amino acid residues replaced by the [MASK] token applied to the given test amino acid sequence representation to generate the library of synthetic peptide or protein sequences.

42.-46. (canceled)
